(19) United States
(12) Patent Application Publication
(10) Pub. No.: US 2009/0112305 A1
(43) Pub. Date: Apr. 30, 2009

(54) Stent-graft prosthesis

(76) Inventors: Helmut Goldmann, Tuttlingen (DE); Herbert Imig, Berlin (DE); Michael Morlock, Hamburg (DE); Anne Gebert, Hamburg (DE)

Correspondence Address: THE NATH LAW GROUP, 112 South West Street, Alexandria, VA, 22314, US

(21) Appl. No.: 11/988,918

(22) PCT Filed: Jul. 19, 2006
(86) PCT No.: PCT/EP2006/007097

§ 371 Date: Jan. 17, 2008

(30) Foreign Application Priority Data
Jul. 19, 2005 (DE) 10 2005 034 617.0
Apr. 27, 2006 (DE) 10 2006 020 687.8

(51) Int. Cl. A61F 2/06

(52) U.S. Cl. 623/1.13; 623/1.15; 623/1.2; 623/1.42; 623/1.35

(57) ABSTRACT

The invention relates to a stent-graft prosthesis with a toroidally expandable sleeve (34) which has at least two opening attachments (36 and 37) and is made of flexible material for internal lining of an arterial aneurysm (1) and, where appropriate, with an aortic insert (23) associated with the sleeve (34), in the form of a vascular prosthesis with expandable supporting structures (39, 40) for fixing the aortic insert (23) in the sleeve (34) in the region of the opening attachments (36 and 37) thereof. The sleeve is designed as foldable toroid. The shape of the sleeve in the unfolded state is adapted to the shape of an aneurysm.

The invention relates to a stent-graft prosthesis with a toroidally expandable sleeve which has at least two opening attachments and is made of flexible material for internal lining of an arterial aneurysm and, where appropriate, with an aortic insert associated with the sleeve, in the form of a vascular prosthesis with expandable supporting structures for fixing the aortic insert in the sleeve in the region of the opening attachments thereof.

A stent graft is an implant which remains permanently in the body and which is deployed inside a vessel, i.e. by means of a catheter for the treatment of abdominal aortic aneurysms (AAA) or thoracic aortic aneurysms (TAA). The intention of implantation of a stent graft is to enable the implant to be permanently anchored in the blood vessel, thus ensuring exclusion of the aneurysm from the bloodstream.

Known stent grafts consist of various graft materials to whose outside or inside, or else incorporated between two material layers, metallic stent structures of differing configuration are attached. Frequently present proximally and/or distally is an uncovered stent and/or hooks which enable anchoring. Reference may be made to the following publications: U.S. Pat. No. 6,517,570 B1, WO 04/037116, WO 01/49211, WO 01/21102 A1, EP 1 086 663 A1, U.S. Pat. No. 6,203,568 B1, WO 00/33769 A1, EP 0 947 179 A2, U.S. Pat. No. 5,938,696, WO 99/29262 A1, WO 98/44870 A1, WO 98/53761 A1, EP 0 893 108 A2, U.S. Pat. No. 6,361,556 B1.

More recent patent applications are concerned with designs intended to avoid the known problems of stent grafts. Pockets are attached to the stent grafts and are intended, filled with incompressible agents, to bring about the expansion of the sleeve of the stent graft (cf. US 2003/0074058 A1 and WO 03/032869 A1). Also known are inflatable cuffs or fluid-type chambers which bring about the expansion of the stent graft by filling with a liquid (US 2002/0116048 A1, WO 99/39662, WO 03/053288 A1). US 2003/0074048 A1 describes a stent graft having an inner impervious sleeve and an outer pervious sleeve with the aim of releasing serum through the pervious sleeve into the sack of an aneurysm.

A further design comprises a stent graft having a port in the wall, through which embolizing materials can be introduced into the aneurysm (US 2002/0169497 A1 and US 2003/0014075 A1). U.S. Pat. No. 6,729,356 B1 discloses a toroidal membrane which is fixed to the prosthesis and which can be filled with blood or other substances. U.S. Pat. No. 6,729,356 B1 discloses a seal in the proximal and distal region which can be introduced separately from the prosthesis. In this case, the prosthesis is connected to the membrane with spikes or spring hooks. Filling material can be introduced into the prosthesis sack and then hardens. There is formation of a barrier against the pressure and a fixing of the prosthesis. WO 04/037116 describes a double-wall prosthesis which has an outer membrane which is expanded elastically. The filling between stent graft and outer, tight membrane is effected with blood or other fillers which can be administered by means of a catheter. A similar possibility is provided by US 2004/0098096 A1. In this case, the sleeve consists of elastic material which expands elastically under the pressure of the inflowing liquid, e.g. blood, and makes contact with the inside of the aneurysm. The possibility of forming bifurcations is also described therein. WO 2004/004603 A1 provides, in the case of an abdominal aortic aneurysm (AAA), for the provision of two aortic inserts which have parallel courses in the aneurysm and then divide at the bifurcation into the leg arteries.

The invention is based on the object of minimizing the drawbacks of conventional stent grafts. It is intended to avoid endoleaks, especially through retrograde collateral arterial flow, migration of the stent graft.

The invention is characterized in that the sleeve is configured as foldable toroid and is adapted to the shape of an aneurysm. The sleeve preferably has a nonelastic pressure-independent volume expandability which corresponds at least to the volume of the aneurysm. At least in the central region of the toroid it is possible for the sleeve to be formed from a substantially non-elastically extensible material. The sleeve may consist completely of elastic material if the sleeve material has in the unextended state at least the size of the aneurysm. The sleeve is able in the filled state to assume a shape corresponding to the internal shape of the aneurysm. The sleeve is able to unfold, owing to the arterial blood pressure, without developing a counter pressure. Since the shape of the sleeve simulates that of an aneurysm, the sleeve is able to make contact at all points on the inner wall of the aneurysm. This results firstly in the sleeve being securely held in the aneurysm, and secondly collaterial arteries are securely closed, making it possible to avoid endoleaks, especially a retrograde collateral arterial flow. Since the sleeve already has the shape of an aneurysm and can be unfolded without pressure, the entire arterial pressure is applied as contact pressure. Thus, no counter pressure generated by the sleeve wall needs to be overcome, as is the case with sleeves able to expand elastically under pressure.

The sleeve is normally associated with an aortic insert which can be inserted into the sleeve. However, the sleeve can also be inserted on its own, i.e. without aortic insert. Separate implantation is indicated in particular when the priority is to stabilize the blood pressure rapidly when an aneurysm has already perforated. The opening attachments which correspond to the proximal and distal ends of the aneurysm taper toward the lumen of the toroid and are adapted to the internal diameter of the aorta in these regions. The stent-graft prosthesis of the invention, especially the sleeve, can be kept in stock in various sizes and shapes in order to take account of the respective size conditions prevailing. Implantation of the sleeve without aortic insert is indicated when the primary concern is to seal the attachments of the collaterals in order to prevent blood flowing back out of the collaterals into the aneurysm. Blood then continues to flow through the volume of the aneurysm within the sleeve.

As already mentioned, the sleeve has a volume expandability without pressure, corresponding at least to the volume of the aneurysm, with the dimensions of the sleeve preferably being greater than the size of the aneurysm. Owing to such an oversizing of the sleeve in the aneurysm, the sleeve may exhibit folds when making contact with the inner wall of the aneurysm. However, this is not a problem. In practice, the sleeve may have up to 5 times, in particular 1.1 to 2 times, the size of an aneurysm.

It is advantageous for the sleeve to be formed from a material which is at least partly foldable. Since it is usually advanced intraarterially with the aid of a catheter as far as the aneurysm and deployed there, it can easily be folded together on the inner lumen of the catheter and then unfolded. In a preferred embodiment, the sleeve material is designed to be elastically extensible at least at the transition of the body of the toroid as far as the opening attachments. It is possible thereby for the sleeve to make contact with the inside of the aorta free of folds in the region of the opening attachments, whereby sealing fixing to the inner wall of the aorta, in particular with the aid of stents, is facilitated. The transition from the elastic material in the region of the opening attachments to a nonelastic material in the region of the volume of the toroid may be abrupt through the nonelastic material being fixed on the elastic material and preferably being folded in the region of the fixing in order to make expansion possible. However, continuous transitions are also possible, for example through longitudinal sections made out of nonelastic material alternating with longitudinal sections made of elastic material, and having gradual transitions in the width, in the peripheral direction of these regions. Suitable elastic material is biocompatible rubber, such as silicone rubber, or else elastic polyurethane. In the case of elastically extensible material, especially in sleeves which consist completely of elastically extensible material, wall sections differing in extensibility can also be obtained by differing wall thicknesses. A toroidal sleeve made of elastic material can be produced by coating an inflatable toroidal core, for example a balloon, with a solution of an elastic synthetic material, for example polyurethane.

In a further embodiment, the sleeve has a rough inner surface. The inner surface may be in particular mechanically roughened. It is particularly advantageous for adhesion of the wall of the sleeve to be avoided through the roughness of the inside of the sleeve and the handling of the sleeve to be improved overall.

The sleeve may further have a smooth outer surface. The invention provides in particular for the sleeve to be provided on its outer surface with a lubricant, in particular in the form of a coating. The lubricant may be for example glycerol.

In a preferred embodiment of the invention, the sleeve is provided on its outside with an adhesive for bonding and permanent sealing on the inner wall of the aneurysm. Normally, a stent-graft prosthesis is anchored more or less mechanically in an aneurysm through its balloon-like or toroidal configuration. Adhesion between sleeve and inner wall of the aneurysm leads to an active fixation and sealing. Suitable and preferred adhesives are biological adhesives, in particular those connected with blood clotting. Examples of suitable adhesives are fibrin adhesives and thrombin adhesives. Time control of the activity of the adhesive is possible, so that the adhesive action occurs only after a predetermined time. Thus, the adhesive layer may be masked with a biodegradable protective layer, thus making it possible to prevent the adhesive power being prematurely active. It is possible to provide for the masking a gel which simultaneously acts as initial lubricant and which favors the unfolding of the sleeve. With combinations of sleeve and aortic insert, it is further advantageous for the inside of the sleeve and/or the outside of the aortic insert to include agents which promote blood coagulation. It is in turn possible and advantageous for such agents to be present as coating. Blood flowing into the space between the sleeve and aortic insert very quickly solidifies through blood clotting and ensures stable conditions.

It is advantageous for the sleeve to be, at least at the time of implantation, preferably permanently, designed to be fluid-tight. This is usual and also expedient for good functioning. Suitable materials for the sleeve are silicone rubber, polyurethane, polyethylene and polytetrafluoroethylene. In a preferred embodiment, the sleeve consists of polyurethane. The polyurethane is in particular an aliphatic polyurethane which is preferably linear. The sleeve normally has a wall thickness in the range from 0.05 to 2 mm. For special cases, it is possible to configure the material of the sleeve in such a way that it is fluid-tight only initially, in a similar way to a vascular prosthesis. Thus, the sleeve may also be formed of textile material, in particular a knitted or woven fabric, which is initially sealed with an absorbable impregnating agent. It is in fact possible to provide for a microfine reticulated supporting structure which is made of a non-absorbable material and which is sealingly coated with absorbable material to be coated on the sleeve. Such a reticulated structure may likewise be textile in nature. Textiles or other porous structures make it possible for biological tissue to grow through and thus make additional anchoring possible.

As already mentioned above, the sleeve and the aortic insert associated therewith may be present separately. This embodiment makes it possible for the two parts of the stent-graft prosthesis to be implanted sequentially in time, i.e. first the sleeve and then the aortic insert. In this embodiment, separate fixing means are associated with the sleeve and its opening attachments, e.g. stents, in particular those which have additional anchoring elements for anchoring to the inner wall of the aorta. Since the sleeve is then already filled with blood and has made contact with the inner wall of the aneurysm, the aortic insert which is to be implanted subsequently may have a covering which is blood-tight (for example made of nonwoven polytetrafluoroethylene), because it is then no longer necessary for blood to flow back through the wall or the covering of the aortic insert into the space between sleeve and aortic insert. In this embodiment, the aortic insert can be sealingly connected at its free ends to the opening attachments of the sleeve. This can take place by pressing the free ends of the aortic insert flatly and tightly on the inner wall of the sleeve in the region of the opening attachments. It is also possible to provide additional sealing and adhesive means. Suitable examples are foams or nonwovens made of collagen or polyurethane.

It is also possible in a known manner for the stent-graft prosthesis to be designed for implantation in an aneurysm where the artery has a bifurcation, as is frequently the case with abdominal aortic aneurysms (AAA). Correspondingly, the sleeve and, where appropriate, the aortic insert then have two distal opening attachments or openings.

For most applications, it is sufficient for the aortic insert already to be sealingly connected at its free ends to the opening attachments of the sleeve before the stent-graft prosthesis is implanted. A chamber is then formed between sleeve and aortic insert and can increase in size after implantation. In this embodiment, the wall or the covering of the aortic insert is pervious to blood at least initially, so that the chamber can be filled, during or after the implantation, by blood flowing through under the arterial blood pressure, and the sleeve makes contact with the inner wall of the aneurysm. When the chamber is filled, and the blood coagulates or clots, the aortic insert is automatically sealed. It is possible to use as aortic insert for example a commercially available vascular prosthesis. This may also be pleated outside the terminal fixing regions. In this embodiment, the chamber between sleeve and aortic insert may in turn comprise means which favor blood coagulation. This can in turn be provided in the form of coatings, or in the form of powdered or liquid additions.

If the aortic insert and sleeve are already connected together, the fixing of the stent-graft prosthesis in the region of the aneurysm can take place in a manner known per se, for example by proximal and/or distal stents. These may be self-expanding or, for example, expandable with a balloon. The sleeve itself may have on its outside fixing means with which it is fixed to the inner wall of the aorta. These fixing means may be present in the form of claws or hooks which are also able to grip through the wall of the sleeve from the inside to the outside.

If the sleeve and aortic insert are separate, then additional fixing means are provided for fixing the aortic insert on the inside of the opening attachments of the sleeve. These may in turn be designed in the form of claws or hooks. The stents may in addition to their expandability also have such fixing means which grip through the wall of the aortic insert into the sleeve. In a preferred embodiment, the inside of the sleeve and the outside of the aortic insert have in the fixing region elements of a touch and close fastener (hooks and loops) which mutually interlock after the implantation. A further possibility is to make the contact areas between sleeve and aortic insert rough and/or finish them with adhesive connecting means.

Further features of the invention are evident from the following description of preferred embodiments in conjunction with the drawing and the dependent claims. It is possible in this connection for the individual features each to be implemented alone or in combination with one another.

### DESCRIPTION OF FIGURES

The drawing shows

FIG. 1: an abdominal aortic aneurysm into which a sleeve according to the invention is inserted,

FIG. 2: the embodiment of FIG. 1, where the sleeve is fixed with stents,

FIG. 3: an abdominal aortic aneurysm with inserted sleeve which has at the proximal end fixing means for fixing an aortic insert,

FIGS. 4a, b, c and d: various fixing elements,

FIGS. 5a and b: fixing elements according to another embodiment,

FIG. 6: an abdominal aortic aneurysm with a sleeve and aortic insert combination according to the invention,

FIG. 7: a sleeve which is folded together and disposed in a catheter in the state for insertion into an aneurysm.

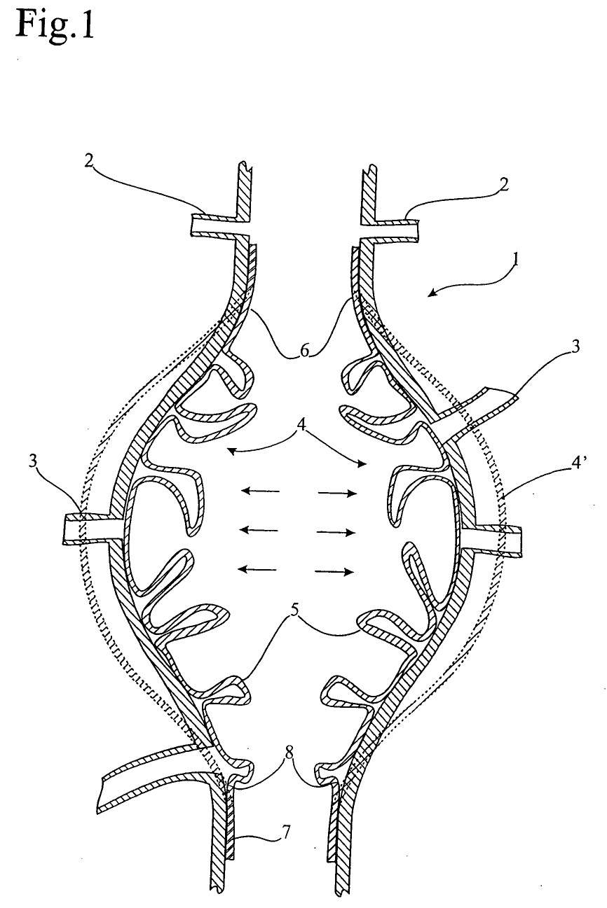

The various embodiments are shown in diagrammatic representation in the drawing. The abdominal aortic aneurysm (AAA) depicted in FIG. 1 is located below the origins of the renal arteries. The arteries 2 which branch off to the two kidneys are unaffected by the aneurysm and must remain open when a stent-graft prosthesis is deployed. By contrast, collateral artery branches 3 which branch off in the region of the aneurysm can be closed. A sleeve 4, which has a toroidal configuration, can be folded together and consists of polyurethane is inserted into the abdominal aortic aneurysm 1. The sleeve has a wall thickness of about 200 μm. The sleeve extends over the entire length of the aneurysm and extends into healthy proximal and distal sections of the abdominal arteries. The sleeve 4 has a toroidal shape in the state filled without pressure and is distinctly oversized, i.e. has greater dimensions, to form folds 5 within the aneurysm (the size of the folds 5 depicted in FIG. 1 is exaggerated). This means that the sleeve would be able in this region also to fill completely a larger or enlarging aneurysm. The full size of the sleeve 4′ is shown shaded in FIG. 1. The sleeve has in the proximal and distal region opening attachments 6 and 7 which are distinctly tapered by comparison with the central region of the sleeve and have only a slightly conical shape. The opening attachments 6 and 7 are adapted in diameter to the internal diameter of the abdominal artery. The sleeve consists in the region of the opening attachments 6 and 7 of elastically extensible material which undergoes a transition into a flexible nonelastic material after an indicated limit 8. The transition may be designed to be gradual. Thus, whereas the sleeve is elastically expandable in the region of the opening attachments and can make sealing contact with the inner wall of the artery with suitable fixing elements, the central section accounting for the actual volume of the sleeve need not be designed to be elastically extensible and, owing to its greater dimensions, is pressed against the aneurysm wall to form folds 5 under the action of the arterial blood pressure (cf. arrows).

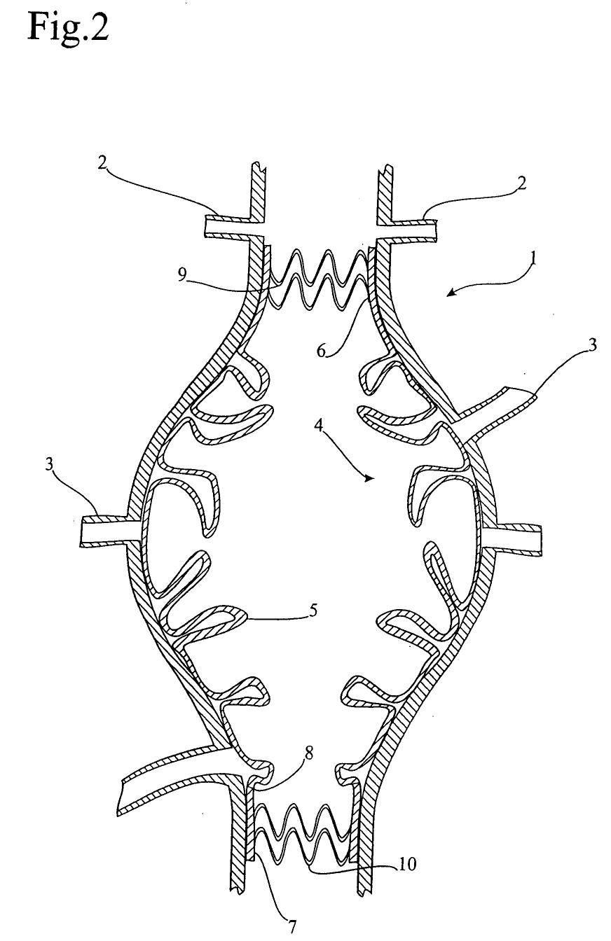

FIG. 2 shows the same embodiment in which a proximal and distal stent 9 and 10 is employed as fixing means on the proximal and on the distal opening attachment 6 and 7 of the sleeve and is either designed to be radially outwardly resilient or is expanded by mechanical means in order to press the sleeve against the inner wall of the artery and fix it there. The stents have an axial length which are approximately the size of the axial length of the opening attachments 6 and 7.

The sleeve shown in FIG. 2 can remain in the fixed state depicted in FIG. 2. It is additionally possible to provide for a subsequent implantation of an aortic insert.

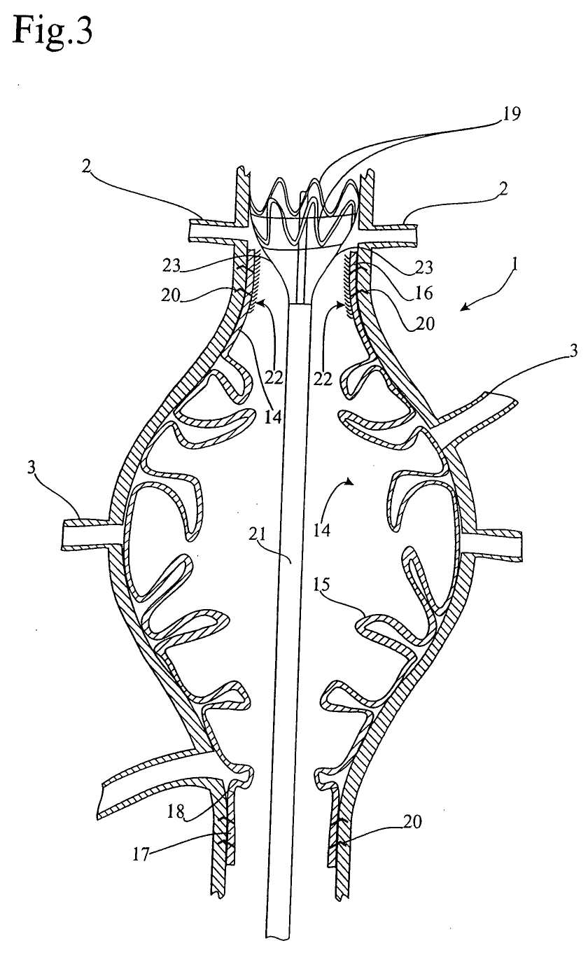

FIG. 3 in turn shows the preparation for fixing an aortic insert for an abdominal aortic aneurysm. A sleeve 14 has substantially the same design as the embodiments shown in FIGS. 1 and 2. Corresponding parts have reference numbers increased by the number 10. At its proximal and distal opening sections 16 and 17, the sleeve 14 grips with the aid of small hooks 20, which are embedded in the sleeve material, to the aortic wall. In addition, a stent 19 is provided in the proximal region and is connected to the upper end of an aortic insert 23 and, after deployment of the aortic insert, presses it together with the sleeve against the inner wall of the aorta. The aortic insert is shown in FIG. 3 in a state in which it is still mostly disposed in a tubular deployment instrument 21.

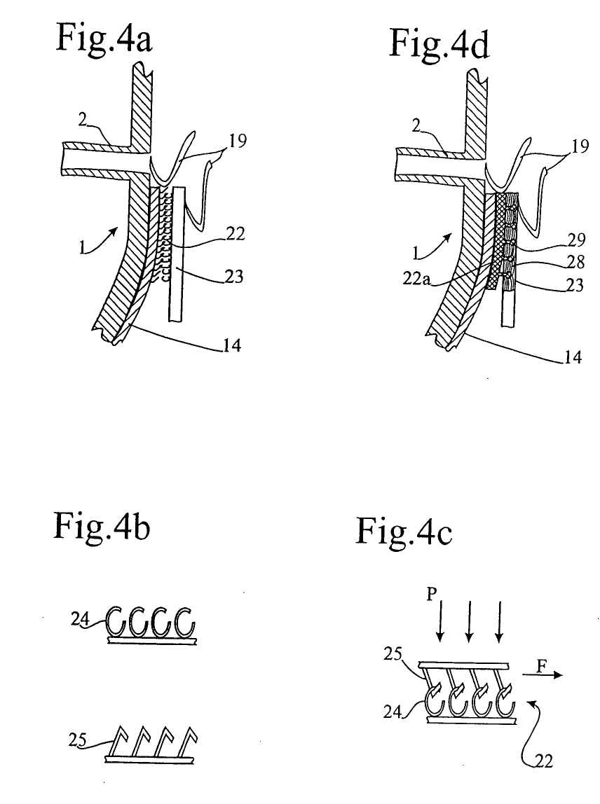

Flatly disposed elements of a touch and close fastener 22 are located on the inside of the proximal opening section 16 of the sleeve 14, and the counterparts thereof are disposed on the outside of the proximal end of the aortic insert 23, as indicated in FIG. 4a. The elements of the touch and close fastener 22 consist of loops 24 and hooks 25 as depicted on a larger scale in FIG. 4b. FIG. 4c shows the engagement of the elements 24 and 25 to form the touch and close fastener 22. When an aortic insert which has correspondingly designed touch and close fastener elements on its outer wall is inserted, it is forced at its proximal end, for example by balloon dilatation, against the inner wall of the opening section 16, thus permanently closing the touch and close fastener 22 and securing where appropriate by a stent 19. It is likewise possible to provide a corresponding touch and close fastener, or else solely a stent, in the distal region. It is also possible to provide the textile wall 28 of the aortic insert 23 as part, specifically as loop part or flange part, of a touch and close fastener 22. The inner wall of the opening section 16 then has the hooks or button parts 29 of the touch and close fastener, as depicted diagrammatically in FIG. 4d. The touch and close fastener 22 or the hook or button part 22a can be designed encircling straight, zig-zag-shaped in the form of parallel strips which are arranged in staggered fashion.

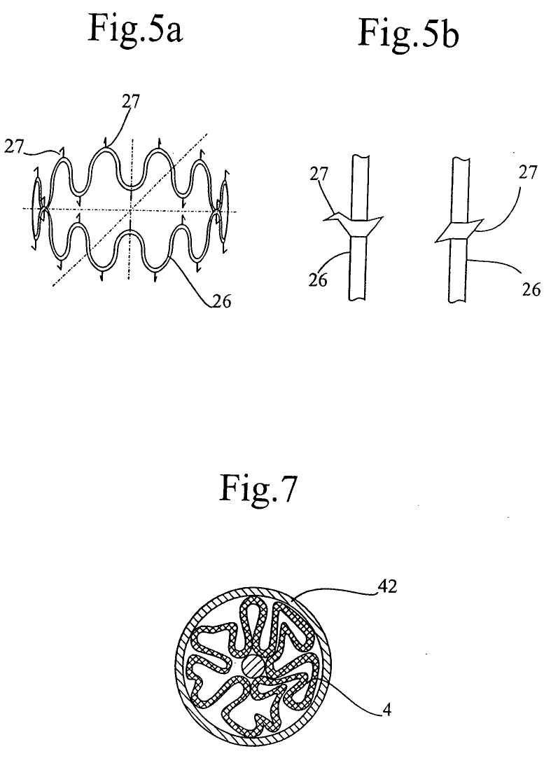

FIG. 5a shows a stent 26 which has a wave-shaped or meander-shaped curvature and which is formed for example from wire which automatically springs back. Compared with a normal stent of this type, the stent shown in FIG. 5a has projections 27 which are directed radially outward at the troughs and crests of the waves and with whose aid the stent is able to grip in a wall, for example of the aortic insert. The gripping can in fact go so far that the projections for example protrude through the wall into the material of the underlying sleeve. Such a stent can also be used for fixing the sleeve in the aortic wall. FIG. 5b shows examples of such projections. They can, when the stent is disposed for example between sleeve and aortic insert, also protrude on both sides, as shown in FIG. 5b. The stents and other fixing means, especially the hooks and projections, may consist of materials known per se. Nitinol, stainless steel 316L or Elgiloy are suitable. Any bare springs may also consist of such materials.

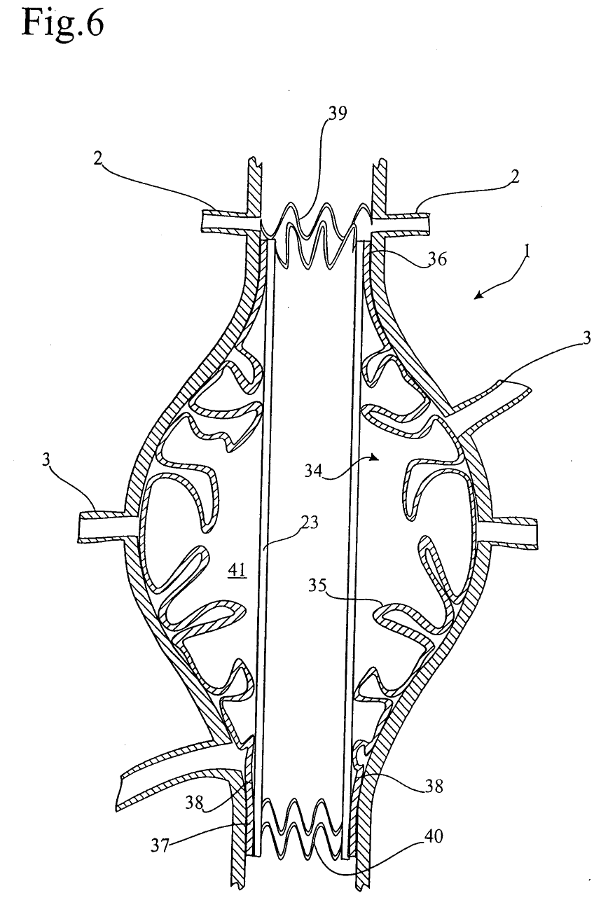

In the embodiment depicted in FIG. 6, a sleeve 34 is inserted into an abdominal aortic aneurysm in the same manner as in the previously described figures. An aortic insert 23 is inserted into the passage of the sleeve between proximal opening attachment 36 and distal opening attachment 37. Its length corresponds to the length of the sleeve 34 deployed in the aneurysm. The aortic insert 23 consists of a knitted textile as usual for vascular prostheses. The knit is not sealingly impregnated, so that blood can pass through the aortic insert 23 into the space or the chamber 41 between aortic insert 23 and sleeve 34 and fill this space in between.

In the embodiment shown in FIG. 6, sleeve 34 and aortic insert 23 are already sealingly connected together before the complete stent graft is implanted. No special fixing means as are provided for the preceding embodiments are therefore necessary. Proximal and distal stents 39 and 40 are sufficient for fixing in the proximal region and in the distal region.

FIG. 7 shows a catheter 42 in cross section, in which the sleeve 4 shown in FIGS. 1 and 2 is arranged with a radial taper. It is possible in the same way for the aortic insert 23 or the combination of sleeves 34 and aortic insert 23 to be arranged in the catheter, because the aortic insert is also compressible. This catheter is used to advance the stent graft intraarterially as far as the aneurysm and then release it.

1-19. (canceled)

20. A stent-graft prosthesis with a toroidally expandable sleeve which has at least two opening attachments and is made of flexible material for internal lining of an arterial aneurysm and, where appropriate, with an aortic insert associated with the sleeve, in the form of a vascular prosthesis with expandable supporting structures for fixing the aortic insert in the sleeve in the region of the opening attachments thereof, wherein the sleeve is designed as foldable toroid, and the shape of the sleeve in the unfolded state is adapted to the shape of an aneurysm.

21. The stent-graft prosthesis as claimed in claim 20, wherein the sleeve has a volume extensibility which corresponds at least to the volume of the aneurysm, with the dimensions of the sleeve being greater than the size of the aneurysm.

22. The stent-graft prosthesis as claimed in claim 20, wherein the sleeve has a size which is up to 5 times the size of the aneurysm.

23. The stent-graft prosthesis as claimed in claim 20, wherein the sleeve is formed at least in the central region of the toroid of a substantially non-elastically extensible material.

24. The stent-graft prosthesis as claimed in claim 20, wherein the sleeve at least at the transition to the opening attachments is formed from elastic material.

25. The stent-graft prosthesis as claimed in claim 20, wherein the sleeve is provided on its outsides with an adhesive for bonding to the inner wall of the aneurysm.

26. The stent-graft prosthesis as claimed in claim 20, wherein the inside of the sleeve and/or the outside of the aortic insert include agents which promote blood coagulation.

27. The stent-graft prosthesis as claimed in claim 20, wherein the sleeve is fluid-tight at least at the time of implantation.

28. The stent-graft prosthesis as claimed in claim 20, wherein the sleeve consists of nonabsorbable material.

29. The stent-graft prosthesis as claimed in claim 20, wherein the prosthesis is formed by the sleeve alone.

30. The stent-graft prosthesis as claimed in claim 20, wherein the sleeve and the aortic insert associated therewith are present separately.

31. The stent-graft prosthesis as claimed in claim 20, wherein the aortic insert can be sealingly connected at its free ends to the opening attachments of the sleeve.

32. The stent-graft prosthesis as claimed in claim 20, wherein the aortic insert has a bifurcation, and the sleeve has three opening attachments in a corresponding manner.

33. The stent-graft prosthesis as claimed in claim 20, wherein the aortic insert is sealingly connected at its free ends to the opening attachments of the sleeve to form an enlargeable chamber between sleeve and aortic insert.

34. The stent-graft prosthesis as claimed in claim 20, wherein a covering of the aortic insert is pervious for blood to pass through.

35. The stent-graft prosthesis as claimed in claim 20, wherein the chamber comprises agents which favor blood coagulation.

36. The stent-graft prosthesis as claimed in claim 20, wherein devices are provided on the outside of the opening attachments of the sleeve.

37. The stent-graft prosthesis as claimed in claim 20, wherein devices are provided on the inside of the opening attachments of the sleeve.

38. The stent-graft prosthesis as claimed in claim 20, wherein the aortic insert can be connected, in particular is connected, via touch and close fasteners to the inside of the opening attachments of the sleeve.

39. The stent-graft prosthesis as claimed in claim 20, wherein the sleeve has a size which is 1.1 to 2 times the size of the aneurysm.

40. The stent-graft prosthesis as claims in claim 20, wherein the whole sleeve is formed from elastic material.

41. The stent-graft prosthesis as claimed in claim 20, wherein the sleeve is permanently fluid-tight.

42. The stent-graft prosthesis as claimed in claim 20, wherein devices for sealing fixing of the prosthesis to the inner walls of the aorta are provided on the outside of the opening attachments of the sleeve.

43. The stent-graft prosthesis as claimed in claim 20, wherein devices for sealing fixing of the aortic insert to the inner wall of the sleeve are provided on the inside of the opening attachments of the sleeve.
